(19) United States
(12) Patent Application Publication
(10) Pub. No.: US 2025/0261959 A1
(43) Pub. Date: Aug. 21, 2025

(54) APPARATUS FOR REMOVING AN ELASTOMERIC TERMINAL PORTION ON A MEDICAL LEAD

(71) Applicant: Medtronic, Inc., Minneapolis, MN (US)

(72) Inventors: Mark T. Marshall, Cape Coral, FL (US); Thomas J. Lulic, Saint Paul, MN (US); Teresa A. Whitman, Dayton, MN (US); Taylor A. Noble, Saint Paul, MN (US); Phillip C. Falkner, Dadeville, AL (US); Megan L. Platner, Minneapolis, MN (US); Mikayle A Holm, Saint Anthony, MN (US); Thai Nguyen, Blaine, MN (US)

(21) Appl. No.: 18/857,301

(22) PCT Filed: Mar. 31, 2023
(86) PCT No.: PCT/IB2023/053261

§ 371 Date: Oct. 16, 2024

Related U.S. Application Data
(60) Provisional application No. 63/363,672, filed on Apr. 27, 2022.

(51) Int. Cl. A61B 17/285; A61B 17/3209; A61B 17/34; A61N 1/05

(52) U.S. Cl. CPC A61B 17/285; A61B 17/3209; A61B 17/3468; A61N 1/057; A61N 2001/0578

(57) ABSTRACT

An apparatus for removing at least one elastomeric terminal component on a proximal end of a medical lead includes a first arm and a second arm. The first arm and the second arm are connected to each other and move in opposition between an open position and a closed position. Opposed mating portions on the first and second arms are configured to retain the proximal end of the lead when the apparatus is in the closed position. An elongate cutting implement enclosed within the second arm includes a cutting blade oriented at an angle such that, when the apparatus is in the closed position, the cutting blade forms a slit in at least a portion of the elastic terminal component.

### BACKGROUND

Implantable medical systems commonly include one or more implantable medical leads coupled to an implantable or external medical device to provide a therapy to the patient. As an example, cardiac systems, such as implantable pacemakers, cardioverter-defibrillators, cardiac resynchronization therapy devices and the like, commonly include an implantable medical device (IMD) such as an implantable pulse generator electrically connected to the heart by at least one transvenous endocardial lead. An endocardial lead provides an electrical pathway between the pacemaker, connected to the proximal end of the lead, and endocardial tissue, which is in contact with the distal end of the lead. Endocardial tissue refers to a specific layer of tissue in the interior of the heart's chambers. Electrical stimulation pulses emitted by the IMD travel through the endocardial lead and stimulate the heart to deliver a prescribed therapy. Other implantable medical systems, such as neuromodulation stimulators, may have leads implanted in other locations of the patient, e.g., brain, spine and the like.

In some patients, it may become necessary to extract and replace an implanted lead. For example, a lead may need to be acutely replaced when unacceptable stimulation thresholds are measured during an implant procedure. A lead may also need to be replaced when the lead fails, or when the endocardial tissue around the lead implantation site becomes infected.

In some examples, if the lead has been implanted for an extended period of time, scar tissue formed around the lead along the intravascular course and at the implantation site in the endocardial tissue can make the extraction procedure more difficult. In such cases, a tubular extraction sheath may be used to track over the length of the lead. The extraction sheath, which may be monitored with fluoroscopy and/or transesophageal echocardiography, is guided within the vasculature of the patient to dissect the fibrotic scar adhesions that prevent lead extraction. In some examples, a distal tip of the tubular extraction sheath may include a rotating cutting element, a laser, a plasma generator, or an electrocautery system to assist in dissecting the adhesions. Once the tubular extraction sheath has been advanced to the implantation site, the lead may be extracted through the sheath and removed from the body of the patient.

In some examples, an inside diameter of an extraction sheath is about 7 French (Fr) (2.3 mm) to as large as 13 Fr (4.3 mm), and smaller extraction sheaths are desirable to prevent injury to the vasculature of the patient. However, the proximal end of pacemaker leads typically have a relatively bulky connector assembly that plugs into, and makes an electrical connection with, the pacemaker. Standard connectors used to connect leads to pacemakers, such as IS-1, IS-4 and DF-4, have an outside diameter of about 14 Fr to 15 Fr (4.6 mm to 5.0 mm). In some procedures, when extracting a lead, the relatively large diameter of the connector can require that the lead be cut near the connector to allow insertion of the lead into the extraction sheath. Once cut, the lead requires additional preparation steps to bind the conductors to a suitable locking stylet or compression coil so that traction may be applied to the lead. Incorrect preparation of the severed conductors can decrease lead tensile strength and increase the chance of lead breakage. Some lead designs can lose substantial lead strength when cut, and cutting the lead to remove the bulky connector may require lead insulation and other protective layers overlying the lead conductor be immobilized by, for example, a ligation suture.

To avoid cutting the lead, some practitioners have adopted a lead removal method that retains the bulky lead connectors, but this method can require use of an extraction sheath with a larger internal diameter to allow passage of the bulky connector. While retaining the connector maintains the structural strength of the lead, the large disparity between the inner diameter of the extraction sheath and the outside diameter of the lead can cause higher complication rates for vascular damage. For example, in some cases the larger gap between the lead outside diameter and the lead inside diameter can pull the vascular wall into the sheath, which can increase the likelihood of a large vascular tear.

### SUMMARY

To maintain lead integrity and strength during a procedure to extract a chronic lead such as, for example a cardiac lead, in some cases it is desirable to leave the lead connectors intact. However, leaving the lead connectors in place on the proximal end of the lead requires an extraction sheath with a larger internal diameter to translate over the relatively large lead connector. To further reduce the diameter of the lead connector, in some cases practitioners employ a scalpel to manually shave off the elastomeric terminal components such as, for example, grip sleeves and seals, on the proximal end of the connector. Removing the elastomeric terminal components reduces the overall diameter of the connectors and the proximal end of the lead, which can make the prepared lead assembly more compatible with an extraction sheath having a preferred size for translation through the vasculature of a particular patient. However, manually shaving away the elastomeric terminal components may potentially leave foreign material at the implant site, which increases patient risk resulting from pocket infections, and the somewhat haphazard shaving procedure may increase risk to the patient, the practitioner, or other operating room personnel.

In general, the present disclosure is directed to a handheld tool that can be used by a practitioner to safely and reproducibly remove one or more elastomeric terminal components from a proximal end of a medical lead, such as, for example, an implanted cardiac lead. In one example, the tool includes opposed arms that may be closed over an elastomeric terminal component on an outer surface of the lead body such as, for example, a silicone grip sleeve, at proximal end of the lead. When the opposed arms of the tool move from an open position to a closed position, an elongate blade housed in the tool is configured to score the elastic terminal component such as, for example, a silicone grip sleeve. The tool may then be translated with respect to the lead to form a slit in the elastic terminal component that extends substantially along a longitudinal axis of the lead. Once longitudinally slit, the elastic terminal component may be peeled away from the lead body in a single piece, or in large multiple pieces, forming fewer shards or shavings. Housing the blade within the body of the tool prevents accidental injury to the practitioner, operating room staff, and patient, and the reduction in small shavings at the implant site can reduce the risk to the patient caused by pocket contamination.

In another example, a portion of the tool includes a port with an optional hypotube having a recessed annular cutting edge. When an elastic terminal component on a proximal end of a medical lead such as, for example, an implanted cardiac lead, is inserted into the hypotube, the annular cutting edge slices the elastomeric terminal component at a diameter slightly greater than the diameter of the lead structure adjacent to the elastic terminal component. The cutting edge engages the elastomeric terminal component, and the tool may then be translated with respect to the lead to strip and peel away the elastomeric terminal component, which remains within a body of the tool to prevent loss in the operating field.

By easily and reproducibly removing the elastomeric terminal components on the proximal end of the lead, the tool of the present disclosure further reduces the overall outside diameter of the lead. For example, reducing the diameter of a cardiac lead can allow use of a smaller extraction sheath during lead extraction procedures. In some cases, removing elastic terminal components such as the grip sleeve and the seal from a cardiac lead can reduce the outside diameter of the connector region on the proximal end of the lead from about 12 French (Fr) (4 mm) to less than about 10 Fr (3.33 mm), or less than about 8 Fr (2.67 mm). This smaller diameter in turn allows the use of a smaller extraction sheath when removing the cardiac lead, which can reduce the likelihood of patient vascular damage.

In one aspect, the present disclosure is directed to an apparatus for removing at least one elastomeric terminal component on a proximal end of a medical lead. The apparatus includes: a first arm and a second arm, wherein the first arm and the second arm each include a first end and a second end, and wherein the first arm and the second arm are connected to each other and move in opposition to each other between an open position and a closed position; wherein the first arm and the second arm include opposed first mating portions and second mating portions configured to retain the proximal end of the lead when the first end of the first arm and the first end of the second arm are in the closed position; and an elongate cutting implement enclosed within the second arm, wherein the cutting implement includes a cutting blade oriented at an angle such that, when the first end of the first arm and the first end of the second arm are moved from the open position to the closed position, the cutting blade is configured to form a slit in at least a portion of the elastic terminal component, and wherein the slit extends along a longitudinal axis of the lead.

In another aspect, the present disclosure is directed to a handheld tool including opposed first and second arms movable about a hinge from an open position to a closed position, wherein the second arm includes an elongate cutting blade within a body thereof, the cutting blade being configured to cut a first elastomeric terminal component on a proximal end of a medical lead and form a slit on the elastic terminal component when the first arm and the second arm are in the closed position, wherein the slit extends along a longitudinal axis of the lead, and wherein the body of the second arm further includes a hypotube with an annular cutting edge configured to form a cut on a second elastomeric terminal component on a proximal end of the lead, wherein the cut formed by the annular cutting edge extends along a direction generally normal to the longitudinal axis of the lead.

In another aspect, the present disclosure is directed to a method for removing a grip sleeve from a proximal end of a medical lead, the method including: retaining the proximal end of the lead between a first arm and a second arm of a tool, wherein the first arm and the second arm are moveable from an open position to a closed position, and wherein at least one of the first arm and the second arm of the tool includes an elongate linear cutting blade; moving the first arm and the second arm into a closed position to engage the linear cutting blade with the grip sleeve on the lead; and translating the lead with respect to the tool such that the linear cutting blade forms a slit in the grip sleeve, the slit extending along a longitudinal axis of the lead.

In another aspect, the present disclosure is directed to a method for removing a seal from a proximal end of a medical lead, the method including: inserting the seal into a hypotube in a tool, wherein the hypotube includes an annular cutting edge; urging the seal against the annular cutting edge such that the seal forms a cut in the seal along a direction normal to a longitudinal axis of the lead; and translating the lead with respect to the tool to strip the seal from the medical lead.

In another aspect, the present disclosure is directed to an apparatus for removing at least one elastomeric terminal component on a proximal end of a medical lead, the apparatus including a first arm and a second arm connected to each other and configured to move in opposition to each other between an open position and a closed position, wherein the first and the second arms include opposed mating surfaces configured to retain the proximal end of the lead when the apparatus is in the closed position, and wherein an elongate cutting implement enclosed within the second arm includes a cutting blade oriented at an angle such that, when the apparatus is in the closed position, the cutting blade slits at least a portion of the elastic terminal component.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a conceptual drawing illustrating an example system that includes a temporary or permanent implantable medical device (IMD) coupled to implantable medical leads.

FIG. 2 is a perspective view of an example of a tool of the present disclosure suitable for removing at least one elastomeric terminal component from a proximal end of a medical lead.

FIG. 3 is an cross-sectional view along line A-A of FIG. 2, which illustrates the hypotube configured to remove a elastomeric terminal component from the proximal end of a medical lead.

FIG. 4 is a perspective view of the tool of FIG. 2 in an open position and retaining a proximal end of a medical lead.

FIG. 5 is a side view of the tool of FIG. 2 in a closed position and retaining a proximal end of a medical lead.

FIG. 6 is a flow chart of a method for removing an elastomeric terminal component such as a grip sleeve from a proximal end of a medical lead using the tool of any of FIGS. 2-5.

FIG. 7 is a flow chart of a method for removing an elastic terminal component such as a seal from a proximal end of a medical lead using the tool of any of FIGS. 2-5. Like symbols in the drawings indicate like elements.

### DETAILED DESCRIPTION

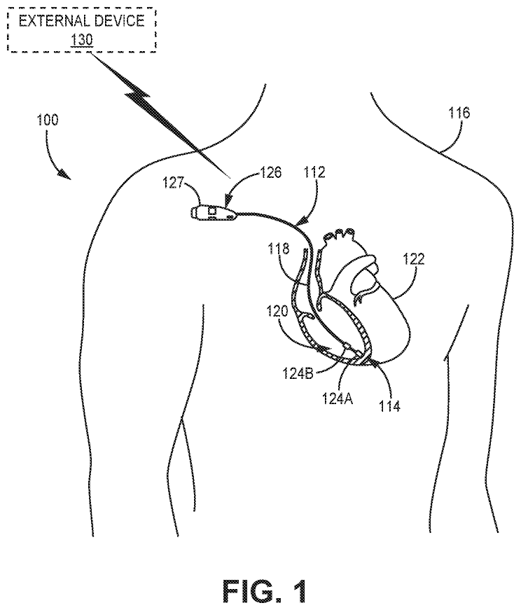

FIG. 1 is a conceptual diagram illustrating a portion of an example implantable medical device system 100. Implantable medical device system 100 may function as a single chamber, e.g., ventricular, pacemaker, as illustrated by the example of FIG. 1 that delivers pacing to a heart 122 of patient 116. Implantable medical device system 100 may be a temporary or permanent pacemaker. In alternative embodiments, however, implantable medical device system 100 may include one or more leads and function a multi-chamber pacemaker, such as a dual-chamber pacemaker or triple-chamber pacemaker that delivers pacing to a heart 122 of patient 116. In some examples, the devices and methods of the present disclosure may be implemented in IMDs other than pacemakers, such as implantable cardioverter-defibrillators (ICDs), implantable cardiac resynchronization (CRT) devices, implantable neurostimulators, or other implantable medical systems that couple to implanted medical leads.

In the example of FIG. 1, implantable medical device system 100 includes one or more implantable medical leads 112 electrically connected to the IMD 126. The implantable medical lead 112 includes an elongated lead body 118 with a distal portion 120 positioned at a target implantation site 114 within the heart 122 such as, for example, a wall within one or more ventricles or atria. The lead 112 may be a unipolar, a bipolar, or a multipolar lead.

A clinician may maneuver the distal portion 120 of the lead 112 through the vasculature of patient 116 to position the distal portion 120 at or near the target site 114. For example, the clinician may guide distal portion 120 through the superior vena cava (SVC) to target site 114 on or in a ventricular wall of heart 122, e.g., at the apex of the right ventricle as illustrated in FIG. 1. Implantable medical device system 100 may include a delivery catheter and/or outer member (not shown), and implantable medical lead 112 may be guided and/or maneuvered within a lumen of the delivery catheter in order to approach target site 114.

The implantable medical lead 112 includes one or a plurality of electrodes. In the example of FIG. 1, the lead 112 includes electrodes 124A and 124B (collectively, “electrodes 124”) configured to be positioned on, within, or near cardiac tissue at or near target site 114. In some examples, electrodes 124 provide pacing to heart 122. The electrodes 124 may be electrically connected to conductors (not shown in FIG. 1) extending through the lead body 118. In some examples, the conductors are electrically connected to therapy delivery circuitry of IMD 126, with the therapy delivery circuitry configured to provide electrical signals through the conductor to electrodes 124. Electrodes 124 may conduct the electrical signals to the target tissue of heart 122, causing the cardiac muscle, e.g., of the ventricles, to depolarize and, in turn, contract at a regular interval. Electrodes 124 may also be connected to sensing circuitry of IMD 126 via the conductors, and the sensing circuitry may sense activity of heart 122 via electrodes 124.

The electrodes 124 may have various shapes such as tines, helices, screws, rings, coils and so on. Again, although a bipolar configuration of lead 112 including two electrodes 124 is illustrated in FIG. 1, in other examples lead 112 may include different numbers of electrodes, such as one electrode, three electrodes, or four electrodes. In configurations in which the lead is a defibrillation lead, the lead may include one or more defibrillation coil electrodes and respective conductors extending through the lead body. In other examples (not shown in FIG. 1), the IMD 126 can be connected to two leads (atrium and right ventricle) or three leads (A, RV, LV), or other electrode or lead configurations.

The configuration of the therapy system 100 illustrated in FIG. 1 is merely one example. In other examples, a therapy system may include epicardial leads, subcutaneous, substernal, and/or patch electrodes instead of or in addition to the transvenous lead 112 illustrated in FIG. 1. Further, the IMD 126 need not be implanted within the patient 116. In examples in which the IMD 126 is not implanted in the patient 116, the IMD 126 may deliver therapies to the heart 122 via percutaneous leads that extend through the skin of patient 116 to a variety of positions within or outside of heart 122.

In one or more examples, IMD 126 includes electronic circuitry contained within an enclosure where the circuitry may be configured to deliver cardiac pacing. In the example of FIG. 1, the electronic circuitry within IMD 126 may include therapy delivery circuitry electrically coupled to electrodes 124. The electronic circuitry within IMD 126 may also include sensing circuitry configured to sense electrical activity of heart 122 via electrodes 124. The therapy delivery circuitry may be configured to administer cardiac pacing via electrodes 124, e.g., by delivering pacing pulses in response to expiration of a timer and/or in response to detection of the activity (or absence thereof) of the heart.

In some examples, the system 100 includes an optional external device 130 such as a programmer. For example, optional external device 130 can be a handheld computing device such as a tablet or a phone, a computer workstation, or a networked computing device. The optional external device 130 can include a user interface that receives input from a clinician, which can include a keypad and a suitable display such as, for example, a touch screen display, or a peripheral pointing device, such as a mouse, via which a user may interact with the user interface. The clinician may also interact with the external device 130 remotely via a networked computing device.

After the implantable medical lead 112 and the electrodes 124 have been temporarily or more permanently implanted in the heart of a patient, the lead 112 and electrodes 124 may need to be removed due to structural defects, infections, or the need to upgrade a pre-existing system. After implantation for extended periods of time (for example, greater than about 1 year), chronic leads may develop a dense fibrotic and sometimes calcific process within the thin-walled venous structures or the endocardial surface of the heart or tricuspid valve, which can make the lead 112 and electrodes 124 difficult to extract. Complex lead extraction is associated with the risk of vascular injury by traction or perforation, causing tamponade, hemothorax, arteriovenous fistula, tricuspid valve disruption, or possibly pulmonary embolism, so simplifying or otherwise improving lead extraction techniques can have significant value for patient safety.

Extraction of medical leads can be performed by a variety of techniques, and in many cases simple traction or traction devices can be used to remove the lead from the body of the patient. However, for chronic leads, various types of extraction sheaths, including mechanical, laser, electrosurgical, rotating threaded tip, and telescoping sheaths may be advanced over the lead and into the vein of the patient to remove the fibrotic scar tissue retaining the lead.

For various types of IMDs 126 (FIG. 1), standard male connector pins of lead 112, such as IS-1, IS-4, DF-4, SQ-1, and the like, have one or more structural features configured to engage a female receptacle in the IMD 126. These standard connector pins have a relatively large diameter compared to the body of the lead 112, and if left intact can require an extraction sheath with a larger diameter to be used. In addition, the standard leads include at least one elastomeric terminal component including, but not limited to, a seal between the connector pin and the body of the lead, and a silicone grip sleeve overlying a portion of the outer surface of the lead body near the proximal end thereof. The elastomeric terminal components further increase the outer diameter of the lead, which requires an even larger extraction sheath be used to move over the connector assembly on the proximal end of the lead. However, as noted above, larger diameter extraction sheaths increase the likelihood of vascular injury, so smaller diameter extraction sheaths are preferred for most patients.

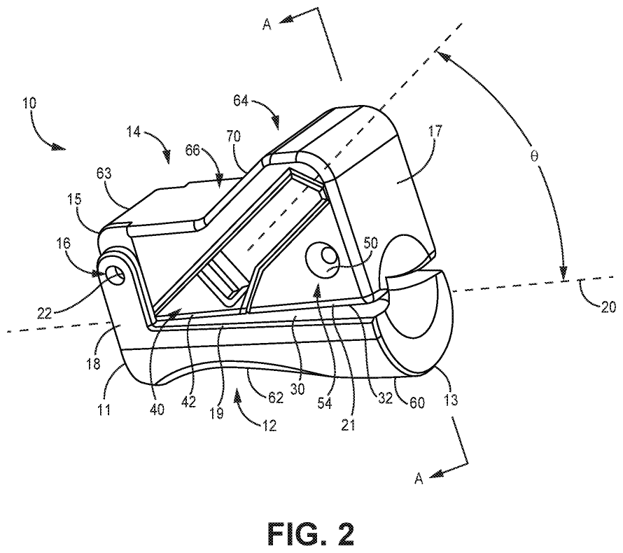

Referring now to FIG. 2, in one aspect, the present disclosure is directed to a handheld tool 10 that can be used by a practitioner to remove the elastomeric terminal components on a proximal end of a medical lead including, but not limited to, an implanted cardiac lead. The tool 10 includes a first arm 12 with a first end 11 and a second end 13. The first arm 12 is connected to a second arm 14, which includes a first end 15 and a second end 17. The first arm 12 and the second arm 14 are configured to move in opposition to each other to retain an end of the medical lead including an elastomeric terminal component.

In the example of FIG. 2, the first arm 12 and the second arm 14 are connected by a hinge 16. In the example of FIG. 2, the hinge 16 is retained within a vertical member 18 extending in a direction generally normal to a longitudinal axis 20 of the tool 10, and includes a pin 22. The first arm 12 and the second arm 14 articulate about the pin 22 such that the first arm 12 and the second arm 14 move in opposition to each other. The first arm 12 and the second arm 14 move about the hinge 16 such that the second end 13 of the first arm 12 and the second end 15 of the second arm 14 move between an open position and a closed position.

In another example (not shown in FIG. 2), the hinge 16 may include members articulating about opposed adjacent edges 19, 21 of the respective first and second arms 12, 14, or may include a continuous flexible hinge portion extending between the edges 19, 21.

In some examples (not shown in FIG. 2), the first arm and the second arm may include one or more interlocking members that form a snap-fit joint. For example, the interlocking members may include one or more annular joints in which a more elastic member on one arm is inserted into a more rigid member in an opposed arm, an arrangement of cantilevered members in one or both of the first and the second arms 12, 14 that insert into corresponding apertures, or may include an arrangement of torsional members on one or both arms that can be deflected to allow insertion of a corresponding member on the opposed arm.

In some examples, the tool 10 further includes an optional biasing member (not shown in FIG. 2) between the first arm 12 and the second arm 14. The biasing member can assist in maintaining the first arm 12 and the second arm 14 in an open position such that the tool 10 can more easily be moved over an elastomeric terminal component on the proximal end of the medical lead, or can be utilized to more forcefully urge a cutting implement (shown in more detail below) into contact with an elastomeric terminal component. The biasing member can vary widely, and in some examples can include a spring, an elastomer, or other biasing member, or a combination thereof.

The first arm 12 includes a substantially planar first mating surface 30, and the second arm 14 includes an opposed substantially planar second mating surface 32. As the first arm 12 and the second arm 14 move from an open position to a closed position, the first mating surface 30 and the second mating surface 32 move in opposition to each other and come together to grip between them an elastomeric terminal component overlying a medical lead. In some examples, the first mating surface 30, the second mating surface 32, or both, may optionally include opposed recessed regions (not shown in FIG. 2, shown in more detail below) shaped to accept and house the proximal end of the medical lead. For example, each of the recessed regions can include a substantially hemispherical cross-sectional shape. When the first arm 12 and the second arm 14 are in a closed position, the opposed recessed regions can move together to more securely grip the elastomeric terminal component on the proximal end of the medical lead.

The second arm 14 further includes a housing 40 that retains therein a cutting implement 42 such that the cutting implement 42 is fully enclosed within the second arm 14. In the example of FIG. 2, which is not intended to be limiting, the cutting implement 42 is an elongate substantially linear blade that is directed toward the first end 11 of the first arm 12, and is oriented at an acute angle θ with respect to the longitudinal axis 20 of the tool 10. As shown in more detail below, as the first arm 12 and the second arm 14 of the tool 10 move into a closed position, the proximal end of the lead is clamped between the opposed mating surfaces 30, 32, and the blade of the cutting implement 42 moves into contact with the elastic terminal component on the lead.

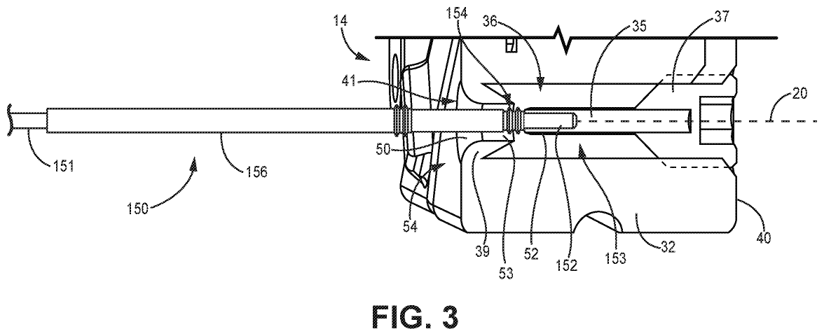

Referring now to FIGS. 2-3, in some examples the housing 40 of the second arm 14 further includes an optional port 54. The port 54 includes a hypotube 35 concentric with the port 54 and recessed within a chamber 36. In the example shown in FIGS. 2-3, the port 54 has a generally conical shape having a wall 50 with a gradually tapering diameter configured to allow insertion of a proximal end of a medical lead. The hypotube 35 terminates in a precision sharpened recessed annular cutting edge 52 sized to the diameter of a connector pin 152 to create an annular cut into an elastic terminal component such as, for example, a seal 154 on the connector 152 as the connector 152 is inserted and pressed into the port 50. A wiper edge 53 strips the cut seal 154 when the lead is pulled out of the port 54, and the annular cutting edge 52 skives off the seal 154 at a diameter slightly larger than the adjacent connector pin 152, which allows the seal 154 to be easily stripped from the lead 150 and retained inside the housing 40.

The first arm 12 and the second arm 14 may be made from a wide variety of materials including, but not limited to, polymeric materials, metals such as stainless steel, titanium, and combinations thereof. In some examples, the first arm 12 and the second arm 14 may be made of a polymeric material, and at least one of the hypotube 35 and the annular cutting edge 52 may be formed from a metal or other material suitable for skiving an elastomeric material such as, for example, a silicone.

In the example of FIG. 2, the first arm 12 and the second arm 14 include optional ergonomic features such that the practitioner can obtain a more secure grip on the tool 10 during procedures for removing an elastic terminal component from a medical lead.

In FIG. 2, the first arm 12 includes an external surface 60 with a first grip portion 62. The first grip portion 62 can have a wide variety of shapes, but in the example of FIG. 2 has a generally arcuate shape configured to engage an index finger of a human hand. In some examples, the first grip portion 62 can be made of soft elastomeric polymeric materials, or may include a textured surface to provide a more secure engagement between the grip surface 62 and the fingers of the practitioner.

The second arm 14 also includes an external surface 64 with a second grip portion 66. The second grip portion 66 can also have a wide variety of shapes, but in the example of FIG. 2 includes a shape configured to engage a thumb of a human hand. The second grip portion includes a substantially planar region 68, and an acutely angled shelf 70 extending upward therefrom and away from the longitudinal axis 20 of the tool 20. In operation, the practitioner may apply force to the shelf 70 to rotate the first arm 12 and the second arm 14 about the hinge 16 from the open position to the closed position. In some examples, the second grip portion 66 can be made of soft elastomeric polymeric materials, or may include a textured surface to provide a more secure engagement between the grip surfaces 66, 68 and the fingers of the practitioner.

FIG. 3 is a cross-sectional view of the body 40 of the second arm 14 that shows a detail of the port 54 and the hypotube 35 therein. The hypotube 35 includes an opening 41 that extends into the body 40 in a direction generally normal to the longitudinal axis 20 of the tool 10. The port opening 41 has a generally conical shape with a wall 50 have a tapering diameter configured to accept a proximal end 153 of a medical lead assembly 150. The proximal end 153 of the lead assembly 150 includes a connector 152, and an elastomeric seal 154 separates the connector 152 from a lead body 151.

In the example of FIG. 3, the hypotube 35 in the tool 10 includes an annular cutting edge 52 configured to grip and cut a seal member on a proximal end of a cardiac lead. The annular cutting edge 52 is supported by a flange 39, which is configured such that the seal 154 inserted into the hypotube 50 is securely urged against the cutting edge 52, which allows the cutting edge 52 to score the seal 154. It should be understood that the shape and size of the flange 39 are merely provided as an example, and the configuration of the flange 39 and the cutting edge 52 can be selected to grip and remove any type of elastomeric terminal component from a medical lead assembly 150.

The body 40 further includes a recessed, well-like region 36 shaped to accept and house a portion of a proximal end of a medical lead. For example, the region 36 may be configured to house the generally cylindrical lead connector 152 adjacent to the seal 154. The region 36 may be shaped and sized to provide a guide for secure insertion of a particular configuration of a connector 152 so that the medical lead assembly 150 can be inserted into the port 54 and the seal 154 can be urged against the annular cutting edge 52.

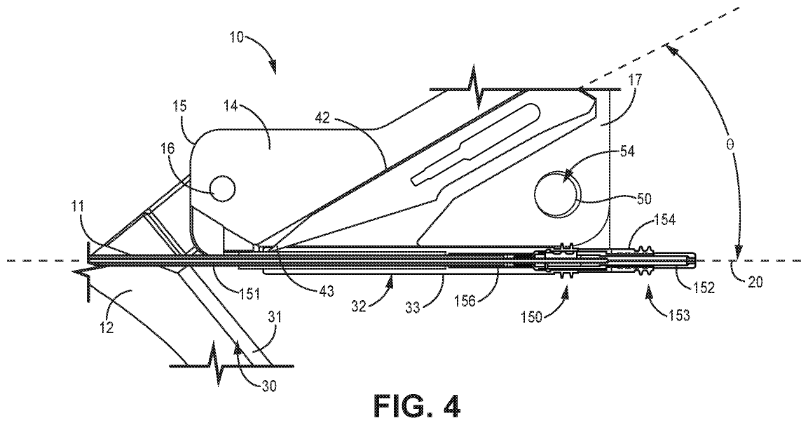

Referring now to FIG. 4, the tool 10 is shown with the first arm 12 and the second arm 14 in an open position. The hinge 16 joins the first end 11 of the first arm 12 to the first end 15 of the second arm 14. The first arm 12 includes a first mating surface 30 with a first recessed region 31, and the second arm 14 includes a second mating surface 32 with a second recessed region 33. The second arm 14 houses a cutting implement 42, which is an elongate substantially linear blade that is directed toward the first end 11 of the first arm 12. The connector 152 protrudes from the second end 17 of the second arm 14. The opposed recessed regions 31, 33 are shaped to retain the lead 150 such that the cutting edge 43 of the cutting implement 43 contacts all or a portion of the length of the silicone grip sleeve 156.

The linear blade is directed toward the first end 15 of the second arm 14 and is oriented at an acute angle θ with respect to the longitudinal axis 20 of the tool 10. In some examples, the angle θ is selected such that substantially all of a cutting edge 43 of the cutting implement contacts the grip sleeve 156 of the medical lead assembly 150 when the first arm 12 and the second arm 14 are closed about the hinge 16 and the medical lead assembly is retained between the first mating surface 30 and the second mating surface 32. The cutting edge 43 is then urged into contact all or a portion of the full length of the grip sleeve 156, and forms a substantially continuous slit in all or a portion of the full length of the grip sleeve. The slit extends along a direction parallel to the longitudinal axis 20 of the tool 10 and the lead assembly 150. In some examples, which are not intended to be limiting, and can depend on the shape and size of the grip sleeve 156, the angle θ is about 10° to about 50°, or about 30°. In some examples, the biasing member (not shown in FIG. 4) between the first arm 12 and the second arm 14, or the body 40 of the second arm 14, may optionally include an adjustment mechanism such as a screw, to allow the cutting edge 43 to penetrate a predetermined distance into the grip sleeve 156.

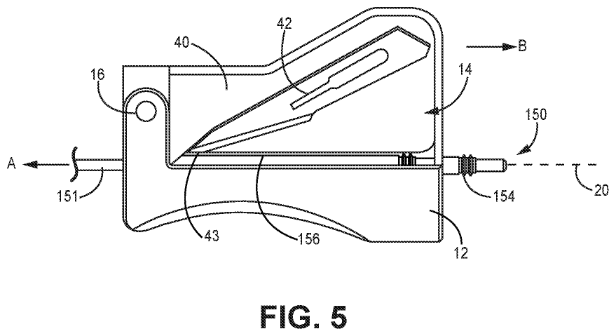

With further reference to FIG. 5, as the first arm 12 and the second arm 14 rotate about the hinge 16 from the open position shown in FIG. 4 to a closed position, the cutting edge 43 is urged into contact with the grip sleeve 156, and scores the grip sleeve 156. With the first arm 12 and the second arm 14 of the tool 10 in the closed position, and the cutting edge 43 remaining in intimate contact with the grip sleeve 156, if the lead body 151 of the lead 150 is pulled in a direction A and the tool 10 remains substantially stationary, the cutting edge 43 forms a longitudinal slit in the grip sleeve 156. Or, if the tool 10 is pulled in a direction B and the lead 150 remains substantially stationary, the cutting edge 43 forms a longitudinal slit in the grip sleeve 156. In other words, as the lead 150 and the tool 10 are translated with respect to each other, the cutting edge 43 forms a substantially continuous slit along a direction parallel to the longitudinal axis 20 of the tool 10.

Referring again to FIGS. 4-5, when the first arm 12 and the second arm 14 are in either an open or a closed position, the proximal seal 154 on the lead may be inserted into the port 54. As the annular cutting edge 52 of the hypotube 35 is urged into contact with the seal 154, the annular cutting edge 52 scores the seal 154, forming a cut that extends in a direction substantially normal to the longitudinal axis 20 of the tool 10 and the lead assembly 150. When the lead 150 assembly is pulled away from the port 54 with the seal 154 contacting the annular cutting edge 52, the scored seal 154 is stripped away from the connector 152. The seal 154 peels cleanly away from the lead assembly 150, and is retained within the body 40 of the second arm 12 to prevent contamination of the surgical field.

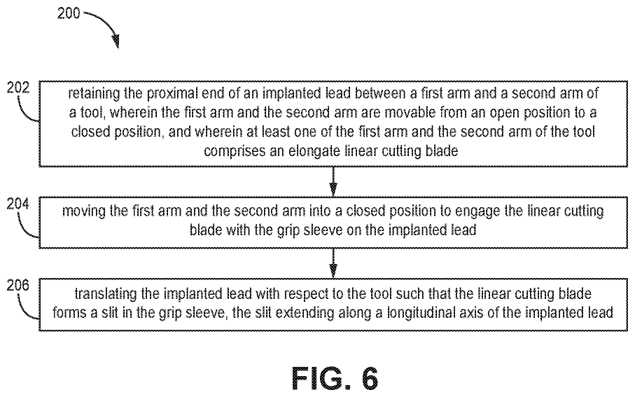

Referring now to FIG. 6, in another aspect, the present disclosure is directed to a method 200 for removing an elastic terminal component such as, for example, a grip sleeve 156, from a proximal end 151 of a medical lead using the tool 10 described above in FIGS. 2-5.

The method 200 includes retaining the proximal end 151 of a lead between a first arm 12 and a second arm 14 of a tool 10 wherein at least one of the first arm and the second arm of the tool includes an elongate linear cutting blade 43 (202).

The method 200 further includes moving the first arm 12 and the second arm 14 into a closed position to engage the linear cutting blade 43 with the grip sleeve 156 on the lead (204).

The method 200 also includes translating the implanted lead with respect to the tool such that the linear cutting blade 43 forms a slit in the grip sleeve 156, wherein the slit extends along a longitudinal axis of the lead (206).

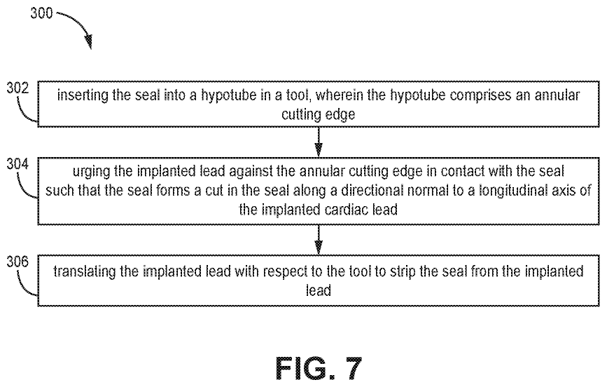

As shown in FIG. 7, in another aspect the present disclosure is directed to a method 300 for removing an elastic terminal component such as, for example, a seal, from a proximal end of a lead using the tool 10 described above in FIGS. 2-5.

The method 300 includes inserting the seal 154 into the port 54 in the body 40 of the second arm 14 such that the seal 154 is urged against the annular cutting edge 52 of the hypotube 35 (302).

The method 300 further includes pulling the endocardial lead from the port 54 with the annular cutting edge 52 contacting the seal 154 such that the cutting edge 52 scores the seal along a direction normal to a longitudinal axis of the lead (304).

The method 300 also includes stripping the seal 154 from the connector on the proximal end of the lead (306).

Various embodiments of the invention have been described. These and other embodiments are within the scope of the following claims.

The following examples are a non-limiting list of clauses in accordance with one or more techniques of this disclosure.

Example 1. An apparatus for removing at least one elastomeric terminal component on a proximal end of a medical lead, the apparatus comprising: a first arm and a second arm, wherein the first arm and the second arm each comprise a first end and a second end, and wherein the first arm and the second arm are connected to each other and move in opposition to each other between an open position and a closed position; wherein the first arm and the second arm comprise opposed first mating portions and second mating portions configured to retain the proximal end of the lead when the first end of the first arm and the first end of the second arm are in the closed position; and an elongate cutting implement enclosed within the second arm, wherein the cutting implement comprises a cutting blade oriented at an angle such that, when the first end of the first arm and the first end of the second arm are moved from the open position to the closed position, the cutting blade is configured to form a slit in at least a portion of the elastic terminal component, and wherein the slit extends along a longitudinal axis of the lead.

Example 2. The apparatus of example 1, wherein the second end of the first arm is connected to the second end of the second arm to form a hinge, and wherein the first arm and the second arm move about the hinge such that the first arm and the second arm pivot in opposition to each other to move the first end of the first arm and the first end of the second arm between an open position and a closed position.

Example 3. The apparatus of any one of example 1 or 2, wherein the medical lead is a cardiac lead.

Example 4. The apparatus of any one of examples 1 to 3, wherein the body of the second arm further comprises a port, wherein the port comprises a hypotube with an annular cutting edge configured to form a cut in at least a portion of the elastomeric terminal component, and wherein the cut formed by the annular cutting edge extends in a direction normal to the longitudinal axis of the lead.

Example 5. The apparatus of any one of examples 1 to 4, further comprising a biasing member between the first arm and the second arm.

Example 6. The apparatus of any one of examples 1 to 5, wherein the first mating portion and the second mating portion comprise opposed first recessed portions and second recessed portions, wherein the first recessed portion and the second recessed portion are shaped to retain the proximal end of the lead when the first end of the first arm and the first end of the second arm are in the closed position.

Example 7. The apparatus of any one of examples 1 to 6, wherein an opening to the hypotube has a substantially conical shape.

Example 8. The apparatus of any one of examples 1 to 7, wherein the first arm comprises a first external surface comprising a first grip portion configured to engage an index finger of a human hand.

Example 9. The apparatus of example 8, wherein the first grip portion has a substantially arcuate shape.

Example 10. The apparatus of any one of examples 8 or 9, wherein the first grip portion comprises a polymeric material.

Example 11. The apparatus of any one of examples 8 to 10, wherein the first grip portion has a surface texture.

Example 12. The apparatus of any one of examples 1 to 11, wherein the second arm comprises a second external surface comprising a second grip portion configured to engage a thumb of a human hand.

Example 13. The apparatus of example 12, wherein the second grip portion comprises a wall with a first portion and a second portion, and wherein the second portion of the wall is angled is configured to engage the thumb.

Example 14. The apparatus of any one of examples 12 or 13, wherein the first portion and the second portion of the wall are each substantially planar.

Example 15. The apparatus of any one of examples 12 to 14, wherein at least a part of the second grip portion comprises a polymeric material.

Example 16. The apparatus of any one of examples 12 to 15, wherein at least a part of the second grip portion has a surface texture.

Example 17. The apparatus of example 5, wherein the biasing member is chosen from a spring, an elastomeric member, or a combination thereof.

Example 18. The apparatus of any one of examples 1 to 17, wherein the first arm and the second arm comprise a material chosen from metals, polymers, and combinations thereof.

Example 19. A handheld tool comprising opposed first and second arms movable about a hinge from an open position to a closed position, wherein the second arm comprises an elongate cutting blade within a body thereof, the cutting blade being configured to cut a first elastomeric terminal component on the proximal end of a medical lead and form a slit in the elastic terminal component when the first arm and the second arm are in the closed position, wherein the slit extends along a longitudinal axis of the lead, and wherein the body of the second arm further comprises a hypotube with an annular cutting edge configured to form a cut on a second elastomeric terminal component on the proximal end of the lead, wherein the cut formed by the annular cutting edge extends along a direction generally normal to the longitudinal axis of the lead.

Example 20. The tool of example 19, further comprising a biasing member between the first arm and the second arm, wherein the biasing member is configured to maintain the tool in the open position.

Example 21. The tool of any one of examples 19 to 20, wherein the hypotube has a substantially conical shape.

Example 22. The tool of any one of examples 19 to 21, wherein the mating surfaces of the first arm and the second arm each comprise a recessed portion shaped to retain the lead.

Example 23. The tool of any one of examples 19 to 22, wherein the first arm comprises a first grip feature configured to engage an index finger of a human hand, and wherein the second arm comprises a second grip feature configured to engage a thumb of a human hand.

Example 24. The tool of example 23, wherein the first grip feature on the first arm has an arcuate shape.

Example 25. The tool of any one of example 23 to 24, wherein the second grip feature on the second arm comprises at least one ledge, and wherein the ledge comprises a substantially planar gripping surface.

Example 26. A method for removing a grip sleeve from a proximal end of a medical lead, the method comprising: retaining the proximal end of the lead between a first arm and a second arm of a tool, wherein the first arm and the second arm are moveable from an open position to a closed position, and wherein at least one of the first arm and the second arm of the tool comprises an elongate linear cutting blade; moving the first arm and the second arm into a closed position to engage the linear cutting blade with the grip sleeve on the lead; and translating the implanted lead with respect to the tool such that the linear cutting blade forms a slit in the grip sleeve, the slit extending along a longitudinal axis of the lead.

Example 27. The method of example 26, wherein the first arm of the tool comprises a grip surface configured to engage an index finger of a human hand, and the second arm of the tool comprises a grip surface configured to engage a thumb of a human hand, wherein the first grip surface and the second grip surface are opposed to each other, and wherein the retaining comprises applying pressure on the opposed first and the second grip surfaces to urge the tool from an open position to a closed position.

Example 28. The method of any one of examples 26 to 27, wherein the first grip surface has an arcuate shape, and the second grip surface comprises an angled ledge.

Example 29. A method for removing a seal from a proximal end of a medical lead, the method comprising: inserting the seal into a hypotube in a tool, wherein the hypotube comprises an annular cutting edge; urging the seal against the annular cutting edge such that the seal forms a cut in the seal along a direction normal to a longitudinal axis of the lead; and translating the implanted lead with respect to the tool to strip the seal from the lead.

Example 30. The method of example 29, wherein the seal, when stripped from the medical lead, remains in a body of the tool.

Example 31. The method of any one of examples 29 to 30, wherein a first arm of the tool comprises a grip surface configured to engage an index finger of a human hand, and a second arm of the tool comprises a grip surface configured to engage a thumb of a human hand, wherein the first grip surface and the second grip surface are opposed to each other, and wherein the method further comprises applying pressure on the opposed first and the second grip surfaces to urge the tool from an open position to a closed position.

Example 32. The method of any one of examples 29 to 31, wherein the first grip surface has an arcuate shape, and the second grip surface comprises an angled ledge.

Example 33. An apparatus for removing at least one elastomeric terminal component on a proximal end of a medical lead, the apparatus comprising a first arm and a second arm connected to each other and configured to move in opposition to each other between an open position and a closed position, wherein the first and the second arms comprise opposed mating surfaces configured to retain the proximal end of the lead when the apparatus is in the closed position, and wherein an elongate cutting implement enclosed within the second arm comprises a cutting blade oriented at an angle such that, when the apparatus is in the closed position, the cutting blade forms a slit in at least a portion of the elastic terminal component.

1. An apparatus for removing at least one elastomeric terminal component on a proximal end of a medical lead, the apparatus comprising:
a first arm and a second arm, wherein the first arm and the second arm each comprise a first end and a second end, and wherein the first arm and the second arm are connected to each other and move in opposition to each other between an open position and a closed position;
wherein the first arm and the second arm comprise opposed first mating portions and second mating portions configured to retain the proximal end of the lead when the first end of the first arm and the first end of the second arm are in the closed position; and
an elongate cutting implement enclosed within the second arm, wherein the cutting implement comprises a cutting blade oriented at an angle such that, when the first end of the first arm and the first end of the second arm are moved from the open position to the closed position, the cutting blade is configured to form a slit in at least a portion of the elastic terminal component, and wherein the slit extends along a longitudinal axis of the lead.

2. The apparatus of claim 1, wherein the second end of the first arm is connected to the second end of the second arm to form a hinge, and wherein the first arm and the second arm move about the hinge such that the first arm and the second arm pivot in opposition to each other to move the first end of the first arm and the first end of the second arm between an open position and a closed position.

3. The apparatus of claim 1, wherein the body of the second arm further comprises a port, wherein the port comprises a hypotube with an annular cutting edge configured to form a cut in at least a portion of the elastomeric terminal component, and wherein the cut formed by the annular cutting edge extends in a direction normal to the longitudinal axis of the lead.

4. The apparatus of claim 1, further comprising a biasing member between the first arm and the second arm.

5. The apparatus of claim 4, wherein the biasing member comprises one of a spring, an elastomeric member, or a combination thereof.

6. The apparatus of claim 1, wherein the first mating portion and the second mating portion comprise opposed first recessed portions and second recessed portions, wherein the first recessed portion and the second recessed portion are shaped to retain the proximal end of the lead when the first end of the first arm and the first end of the second arm are in the closed position.

7. The apparatus of claim 3, wherein an opening to the hypotube has a substantially conical shape.

8. The apparatus of claim 1, wherein the first arm comprises a first external surface comprising a first grip portion configured to engage an index finger of a human hand.

9. The apparatus of claim 8, wherein the first grip portion has a substantially arcuate shape.

10. The apparatus of claim 8, wherein the second arm comprises a second external surface comprising a second grip portion configured to engage a thumb of a human hand.

11. The apparatus of claim 10, wherein the second grip portion comprises a wall with a first portion and a second portion, and wherein the second portion of the wall is angled is configured to engage the thumb.

12. The apparatus of claim 11, wherein the first portion and the second portion of the wall are each substantially planar.

13. The apparatus of claim 10, wherein at least one of the first grip portion or the second grip portion or both has a surface texture.

14. A handheld tool comprising opposed first and second arms movable about a hinge from an open position to a closed position, wherein the second arm comprises an elongate cutting blade within a body thereof, the cutting blade being configured to cut a first elastomeric terminal component on the proximal end of a medical lead and form a slit in the elastic terminal component when the first arm and the second arm are in the closed position, wherein the slit extends along a longitudinal axis of the lead, and wherein the body of the second arm further comprises a hypotube with an annular cutting edge configured to form a cut on a second elastomeric terminal component on the proximal end of the lead, wherein the cut formed by the annular cutting edge extends along a direction generally normal to the longitudinal axis of the lead.

15. An apparatus for removing at least one elastomeric terminal component on a proximal end of a medical lead, the apparatus comprising a first arm and a second arm connected to each other and configured to move in opposition to each other between an open position and a closed position, wherein the first and the second arms comprise opposed mating surfaces configured to retain the proximal end of the lead when the apparatus is in the closed position, and wherein an elongate cutting implement enclosed within the second arm comprises a cutting blade oriented at an angle such that, when the apparatus is in the closed position, the cutting blade forms a slit in at least a portion of the elastic terminal component.

16. The tool of claim 14, further comprising a biasing member between the first arm and the second arm, wherein the biasing member is configured to maintain the tool in the open position.

17. The tool of claim 14, wherein the mating surfaces of the first arm and the second arm each comprise a recessed portion shaped to retain the lead.

18. The tool of claim 14, wherein the first arm comprises a first grip feature configured to engage an index finger of a human hand, and wherein the second arm comprises a second grip feature configured to engage a thumb of a human hand.

19. The tool of claim 19, wherein the first grip feature on the first arm has an arcuate shape.

20. The tool of claim 19, wherein the second grip feature on the second arm comprises at least one ledge, and wherein the ledge comprises a substantially planar gripping surface.
